(19) United States
(12) Patent Application Publication
(10) Pub. No.: US 2009/0198290 A1
(43) Pub. Date: Aug. 6, 2009

(54) Non-Clamping Fastening Mechanism With Anti-Splay Feature

(75) Inventors: Rex W. Armstrong, Cordova, TN (US); Randall N. Allard, Germantown, TN (US); Carlos Gil, Collierville, TN (US); Thomas A. Carls, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 11/961,635

(22) Filed: Feb. 6, 2008

(51) Int. Cl. A61B 17/58

(52) U.S. Cl. 606/305; 606/301

(57) ABSTRACT

A device for attaching an elongate support member to the spinal column includes a head portion and a fastening member. The head portion may have a U-shaped portion defining a transverse passage therethrough. The passage may be sized to receive a portion of the elongate support member. The head portion may have an opening between a pair of spaced apart arm portions, the arm portions having end surfaces and having inwardly facing threads. The device also may include a fastening member comprising a body portion having threads sized to interface with inwardly facing threads on the head portion, the body portion defining a longitudinal axis. The fastening member also may include a flange portion adjacent the body portion. The flange portion may extend outwardly about the longitudinal axis to define a circular flange perimeter. The flange portion may include a transversely extending protruding portion and an angled protruding portion.

### FIELD OF THE INVENTION

The present invention relates generally to the field of anchor mechanisms for securing an implanted elongated member.

### BACKGROUND

Elongated plates and rods may be attached to the spinal column for spinal support or stabilization in the treatment of spinal maladies, such as scoliosis. Patients with infantile or juvenile scoliosis who undergo stabilization treatment via the use of subcutaneous plates and rods may be subject to multiple surgical procedures for adjustment as they grow. This is particularly applicable to patients under the age of 10 years old, where portions of the spinal construct must be periodically replaced with one of greater length to accommodate for continued growth of the spinal column (e.g., to compensate for an increase in the distance between the spinal motion segments). In some instances, such adjustments may be required every six months, thereby requiring multiple surgeries throughout the treatment process.

The devices and methods disclosed herein overcome one or more of the deficiencies in the prior art.

### SUMMARY

In one exemplary aspect, the present disclosure is directed to a device for attaching an elongate support member to the spinal column. The device may comprise a head portion having a U-shaped portion defining a transverse passage therethrough. The passage may be sized to receive a portion of the elongate support member. The head portion may have an opening between a pair of spaced apart arm portions, the arm portions having end surfaces and having inwardly facing threads. The device also may include a fastening member comprising a body portion having threads sized to interface with inwardly facing threads on the head portion, the body portion defining a longitudinal axis. The fastening member also may include a flange portion adjacent the body portion. The flange portion may extend outwardly about the longitudinal axis to define a circular flange perimeter. The flange portion may include a transversely extending protruding portion and an angled protruding portion.

In another exemplary aspect, the present disclosure is directed to a setscrew for a spinal construct having a head portion. The setscrew may include a body portion having threads sized to interface with inner threads on the head portion, the threads extending less than 720 degrees about the exterior of the body portion, the body portion defining a longitudinal axis. It also may include a flange portion adjacent the body portion, the flange portion extending outwardly about the longitudinal axis to define a circular flange perimeter, the flange including a transversely extending protruding portion and an angled protruding portion.

In another exemplary aspect, the present disclosure is directed to an anchor member for attaching an elongate support member to the spinal column. The anchor member may comprise a head portion defining a first longitudinal axis and having a transverse passage therethrough configured to receive a portion of said support member. A setscrew member may be threadably engageable with the head portion in a manner to secure the support member in the passage. The setscrew member may define a second longitudinal axis and may have a transversely extending flange. The flange may have a surface portion configured to interface with the head portion in a manner that applies loading to the head portion in both a longitudinal direction and a transverse direction.

In yet another exemplary aspect, the present disclosure is directed to an implant, comprising an elongate support member sized to span a plurality of vertebrae and an anchor member configured to provide substantially unconstrained axial movement of the anchor member relative to the elongate member. The anchor member may include a bone engaging portion and a head portion connected to the bone-engaging portion. The head portion may have a U-shaped portion defining a transverse passage therethrough sized to receive a portion of the support member. The head portion may have a frusto-conical outer surface portion. The anchor member also may include a setscrew member threadably engageable with the head portion. The setscrew member may define a longitudinal axis and having a transversely extending flange, the flange having a frusto-conical surface portion configured to interface with the frusto-conical outer surface portion of the head portion. The setscrew may be sized to engage with the head portion in a manner that non-rigidly secures the support member in the passage when the flange portion interfaces with the head portion.

Further aspects, forms, embodiments, objects, features, benefits, and advantages of the present invention shall become apparent from the detailed drawings and descriptions provided herein.

### BRIEF DESCRIPTION OF THE DRAWINGS

FIG. 1 is an illustration of a posterior view of a spinal rod system according to one form of the present invention, as attached to a posterior aspect of the spinal column.

FIG. 2 is an illustration of a lateral view of the spinal rod system illustrated in FIG. 1.

FIG. 3 is an illustration of an exemplary anchor member usable in a spinal rod system.

FIG. 4A is an illustration of a cross-sectional view of the anchor member of FIG. 3 with a fastening member disengaged from the head portion.

FIG. 4B is an illustration of a cross-sectional view of the anchor member of FIG. 3 with a fastening member engaged with the head portion.

FIG. 4C is an illustration of an enlarged portion of the cross-sectional view of the anchor member of FIG. 4B.

FIG. 5 is an illustration of an isometric view of an exemplary fastening member.

FIG. 6 is an illustration of a cross-sectional view of the fastening member of FIG. 5.

FIG. 7 is an illustration of another exemplary anchor member usable in a spinal rod system.

FIG. 8 is an illustration of another exemplary anchor member usable in a spinal rod system.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

One exemplary process of accommodating continued growth of a spinal column with a spinal construct includes implanting an oversized elongated member and to non-rigidly secure it along the spinal column, thereby permitting the vertebral column to grow within the range of the construct. In order to non-rigidly secure the elongated member in place, the elongated member may extend through receiver head portions of bone screws, without being clamped to more than one bone screw. This permits the unclamped bone screw to prevent or limit lateral movement of the elongated member and hence the spinal column. Yet, as the spinal column grows in length, the elongated member may axially move through the receiver heads of the unclamped bone screws, thereby accommodating the growth.

In some embodiments, the elongated member may be secured to the bone screws using fastening members, such as setscrews that may be tightened into the receiver head portion. These may be configured to prevent lateral movement of the elongated member from the bone screw, but do not rigidly clamp to engage the elongated member. This permits the elongated member to move along its axis. One such screw is disclosed in commonly-owned U.S. patent application Ser. No. 11/102,167, incorporated herein in its entirety by reference. Because the setscrew does not lie deeply into the receiver head, the setscrew may engage the receiver head portion along only very few full threads, and sometimes only one or two threads. As the applied loads act on these very few engaged threads, the receiver head portion may begin to splay, causing loosening of the setscrew in the receiver head portion. The overall length of the receiver head portion and setscrew can be increased to permit engagement of a greater number of thread turns, but this also increases the overall height of the screw head portions. The setscrew design disclosed herein may reduce the chance of the head portion splaying while still providing a low profile screw.

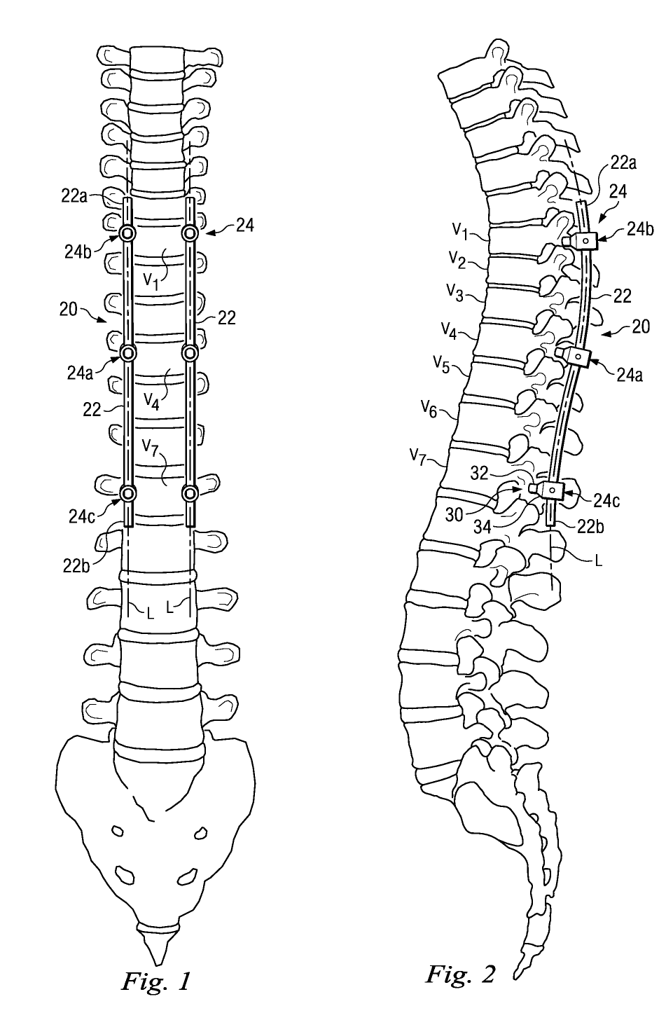

FIGS. 1 and 2 show a stabilization system 20 according to one exemplary aspect of the present invention for stabilizing at least a portion of the spinal column. In the illustrated embodiment, the stabilization system 20 extends across a plurality of vertebral levels including vertebrae V₁-V₇. However, it should be understood that the stabilization system 20 may extend across any number of vertebral levels including two or more vertebrae. Additionally, in the illustrated embodiment, the stabilization system 20 is attached to a posterior aspect of the spine. However, it should also be understood that the stabilization system 20 may be attached to other aspects of the spine, including anterior, antero-lateral, lateral, and/or postero-lateral aspects of the spine. It should be understood that the stabilization system 20 may utilize any number of elongate members 22, including a single elongate member or three or more elongate members. Further, although the stabilization system 20 is illustrated as being used in association with the thoracic region of the spine, it should be understood that the stabilization system 20 may be utilized in other regions of the spine, including the cervical, lumbar, lumbo-sacral and sacral regions of the spine. It should also be understood that the stabilization system 20 can be used in association with fusion or fusionless treatment of the spine.

In the illustrated embodiment, the stabilization system 20 includes a pair of elongate members 22, each extending along a longitudinal axis L and each engaged across a number of vertebral levels via one or more anchor members 24. Here, the elongate members 22 are engaged to the spine by a first pair of anchor members 24a at a location at or near the coronal apex of the thoracic curve (e.g., adjacent vertebra V₄), by second and third pairs of non-clamping anchor members 24b, 24c respectively located one or more levels above the coronal apex (e.g., adjacent vertebra V₁) and located one or more levels below the coronal apex (e.g., adjacent vertebra V₇). Engagement of one or more elongate members 22 to the spine in this manner is sometimes referred to as the Shilla technique. Although the elongate members 22 are specifically illustrated as being specifically engaged to the vertebrae V₁, V₄, and V₇, it should be understood that the elongate members 22 may be engaged to each of the vertebrae V₁-V₇, to every other one of the vertebrae V₁-V₇, or to any number of the vertebrae V₁-V₇ via other layouts or attachment configurations.

According to the exemplary aspect disclosed herein, the stabilization system 20 is configured to provide lateral stabilization or support to the portion of the spine being treated, while at the same time allowing for at least some degree of relative axial movement or motion between the vertebrae to which the elongate members 22 are engaged. In one embodiment, engagement between the elongate member 22 and at least one of the anchor members 24a is fixed or clamped so as to substantially prevent relative axial movement therebetween, and with engagement between the elongate member 22 and at least one of the anchor members 24b, 24c being variable or unclamped so as to allow for relative axial movement therebetween. The combination of fixed and variable engagement between the elongate members 22 and the anchor members 24a-c provides at least some degree of relative axial movement or motion between the vertebrae to which the elongate members 22 are engaged (e.g., vertebrae V₁, V₄, and V₇). In one embodiment, the unclamped anchor members 100 that are variably engaged to the elongate member 22 allow for sliding movement of the anchor members 100 along an axial length of the elongate member 22 (e.g., in a direction generally along the sagittal plane), thereby allowing for relative axial movement or motion between the vertebrae to accommodate for continued growth of the patient's spine.

In the illustrated exemplary embodiment of the invention, the intermediate anchor members 24a are of the fixed or clamped type so as to substantially prevent relative axial movement between the elongate member 22 and the vertebra V₄. Additionally, the outer anchor members 24b, 24c are each of the variable or unclamped type so as to allow for relative axial movement between the elongate member 22 and the anchor members 24b, 24c. Such variable or unclamped engagement between the elongate member 22 and the anchor members 24b, 24c likewise allows for relative axial movement between the elongate member 22 and the vertebrae V₁, V₇, which in turn allows for relative axial movement between each of the vertebrae V₁, V₄ and V₇ (and the intermediate vertebrae V₁, V₂ and V₅, V₆) to accommodate for growth of the patient's spine. Accordingly, the elongate members 22 provide lateral stabilization or support to the portion of the spine being treated, while at the same time allowing for substantially unrestricted or uninhibited axial movement to accommodate for growth of the patient's spine.

In one embodiment of the invention, each of the elongate members 22 is generally configured as an elongate tether, and more specifically as an elongate spinal rod that may have a flexible or semi-flexible configuration and may exhibit resilient or semi-resilient characteristics. Although the elongate members 22 are illustrated as having a rod configuration, it should be understood that the elongate members 22 may take on other configurations as well such as, for example, plates, cables, wires, artificial strands, artificial ligaments, springs, or other types of elongate members known to those of skill in the art that are suitable to stabilize and/or support at least a portion of the spine.

In the illustrated embodiment of FIGS. 1 and 2, the anchor members 24a-c are bone anchors 30 comprising a bone engaging portion 32 structurally configured for engagement with vertebral bone, and a receiver portion 34 structurally configured for engagement with one of the elongate member 22. However, it should be understood that other types and configurations of anchor members are also contemplated as falling within the scope of the present invention including, for example, spinal hooks, staples, various types and configurations of connectors, or other types of anchor members known to those of skill in the art that are suitable for engaging one or more elongate members 22 to the spinal column. In one embodiment, the anchor members 24 are formed of a metallic material such as, for example, titanium, a titanium alloy, stainless steel, a chrome-cobalt alloy, a shape-memory or superelastic material such as nitinol, or other suitable metallic materials know to those of skill in the art. In other embodiments, the anchor members 24 may be formed of a polymer such as, for example, polyester or polyethylene, or from a synthetic material.

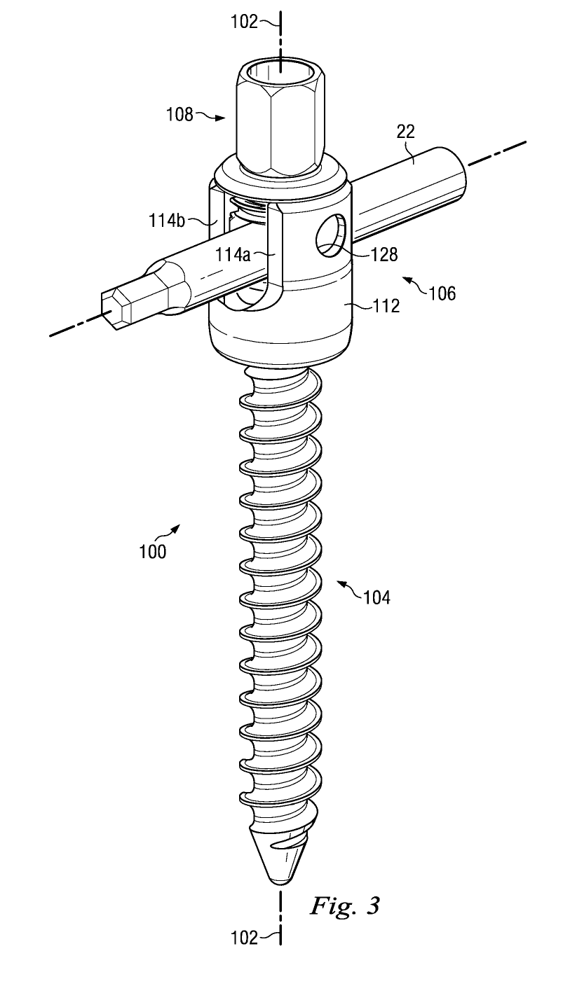

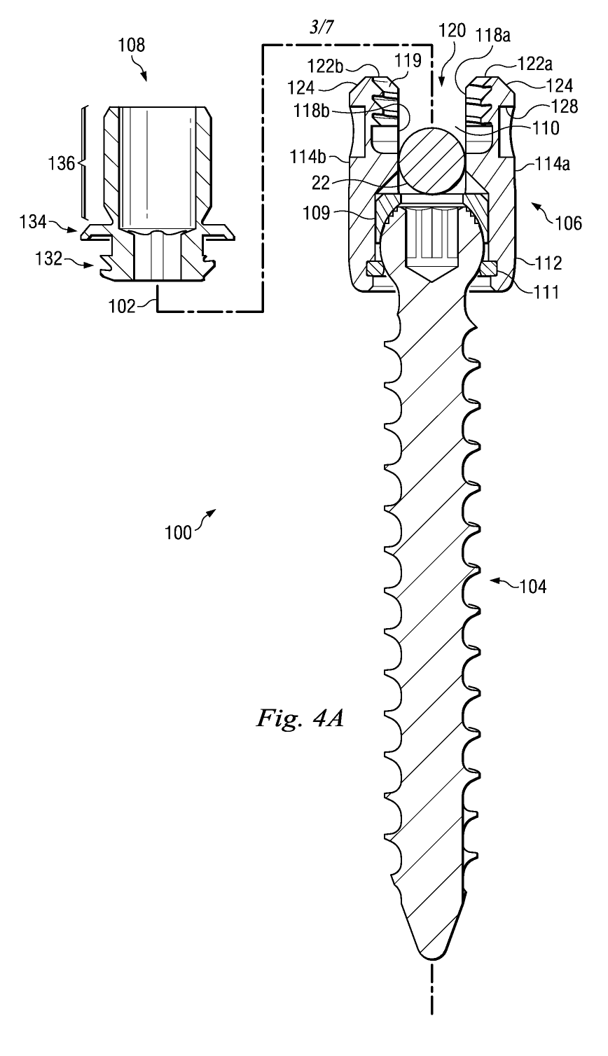

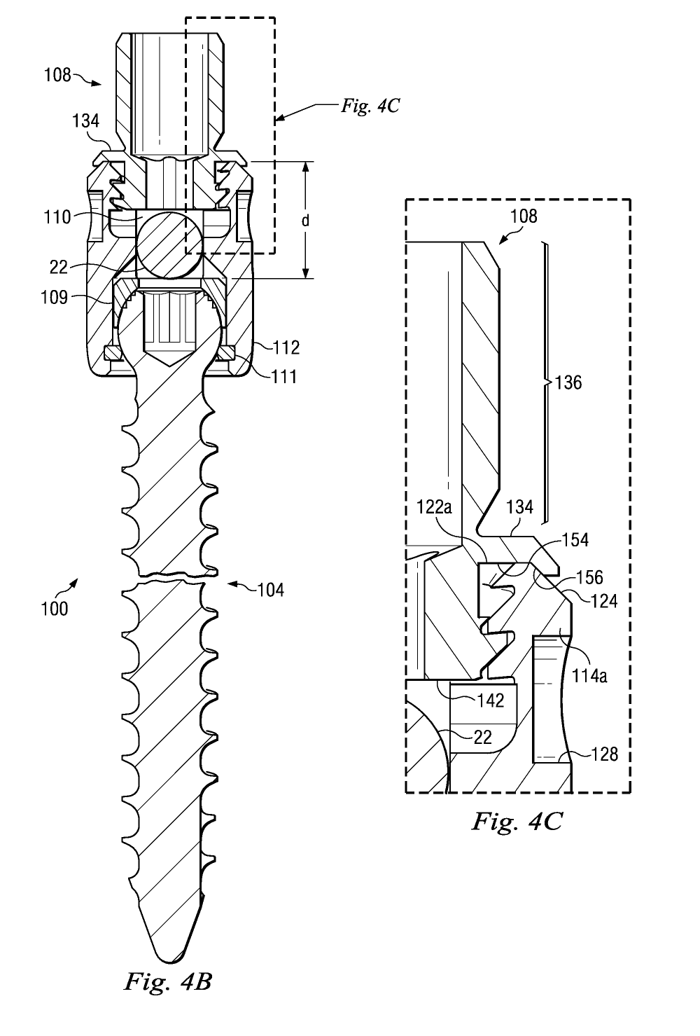

FIGS. 3 and 4A-C show one exemplary embodiment of anchor members 24b, 24c of the variable or unclamped type which are configured to allow for axial movement relative to the spinal rod 22. In the illustrated embodiment, the anchor members 24b, 24c are configured as bone anchors, referenced herein by the numeral 100, extending along a longitudinal axis 102, and more specifically as pedicle screws. However, it should be understood that other types and configurations of bone anchors are also contemplated for use in association with the present invention.

The bone anchor 100 generally includes a threaded shaft portion 104, a head portion 106, and a fastening member 108. In the illustrated embodiment, the bone anchor 100 is a multi-axial screw where the threaded shaft portion 104 and the head portion 106 are formed separately and cooperate in a manner to permit the head portion 106 to move relative to the threaded shaft portion 104. Here, in the example shown, this is accomplished by a crown member 109 and a snap ring 111 configured to assist in securing the head portion 106 and the threaded shaft portion 104 together. However, in other embodiments, alternative multi-axial screws are used and in yet other embodiments, the threaded shaft portion 104 and the head portion 106 are formed integral with one another so as to define a single-piece bone anchor. The fastening member 108 cooperates with the head portion 106 to capture and retain the spinal rod 22 within the head portion 106 of the bone anchor 100 in a variable or unconstrained manner so as to allow for relative axial movement between the spinal rod 22 and the bone anchor 100.

Although the shaft portion 104 is illustrated and described as defining external threads, it should be understood that the shaft portion 104 may alternatively define other features for anchoring to bone such as, for example, barbs, teeth, pivoting gulls, or other suitable anchoring features known to those of skill in the art.

As shown in the exemplary embodiment in FIGS. 3 and 4A-B, the exemplary head portion 106 forms a U-shape having a base portion 112 and a pair of spaced apart arm portions 114a, 114b extending from the base portion 112. This U-shape defines a passage 110 extending transversely through the head portion 106 sized and configured to receive one of the elongate members 22 therein.

In the exemplary embodiment shown, the arm portions 114a, 114b extend substantially parallel to one another and define a pair of spaced apart side surfaces 118a, 118b (FIG. 4A). The transverse passage 110 is preferably configured to provide a relatively close fit between the elongate member 22 and the side surfaces 118a, 118b (FIG. 4A) of the head portion 106, the purpose of which will be discussed below. At least the upper portions of the inner side surfaces 118a, 118b define internal threads 119 for engaging with the fastening member 108. Although a specific configuration of the head portion 106 has been illustrated and described herein, it should be understood that other types and configurations of anchor head portions and other shapes and configurations of the transverse passage are also contemplated as falling within the scope of the present invention.

As best shown in FIG. 4A, the head portion 106 has an open upper end 120 in communication with the passage 110, with ends of the arm portions 114a, 114b defining end surfaces 122a, 122b, respectively, adjacent the open upper end 120. The end surfaces 122a, 122b are offset from the crown member 109, thereby providing the passage 110 with an overall depth d (FIG. 4B).

Adjacent the end surfaces 122a, 122b, the head portion 106 includes an angled outer surface portion 124, best seen in FIG. 4C. This angled surface portion 124 extends entirely about the circumference defined by the arm portions 114a, 114b of the head portion 106, thereby forming a partially frusto-conical shape. In some exemplary embodiments, the angle is about in the range between 30-60 degrees. In other embodiments, the angle is about in the range of 40-50 degrees. Other ranges are contemplated. As shown in FIG. 4C, the fastening member 108 is shaped to correspond with the end surfaces 122a, 122b and the angled outer surface portion 124. Because of the angled surface portion, the end surfaces 112a, 122b do not extend to the greatest outer circumference of the head portion 106. Because of this, in the example shown, the overall surface area of the end surfaces 122a, 122b may be smaller than the thickness of the arm portions 114a, 114b.

The head portion 106 also includes indentations or recesses 128 sized and configured to receive corresponding portions of an instrument or tool therein (e.g., an insertion tool, a driver instrument, a compression instrument).

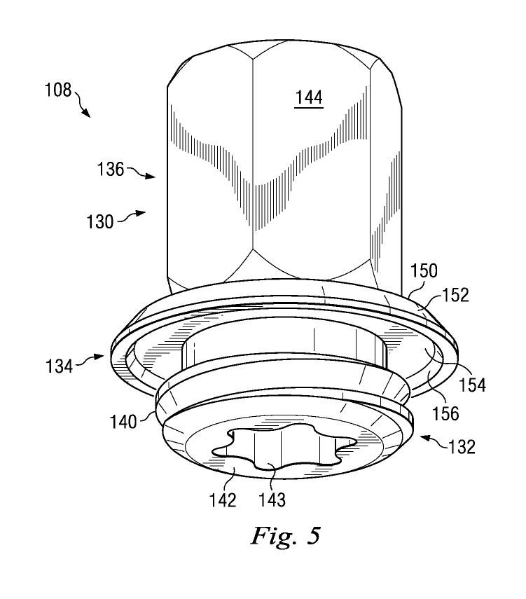

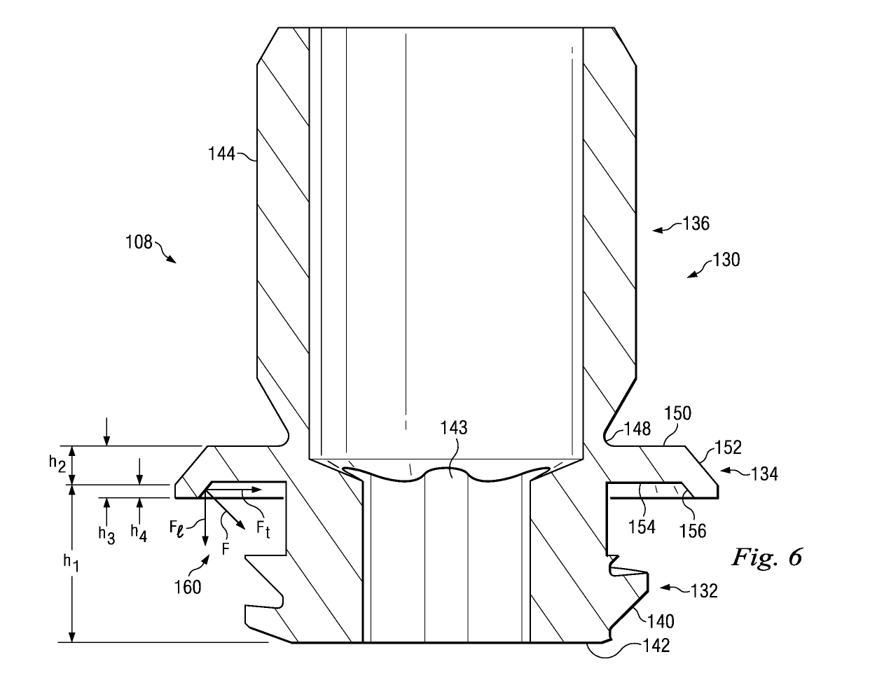

One exemplary fastening member 108 is shown in FIGS. 3-6. FIGS. 5 and 6 show the fastening member 108 independent of other features of the bone screw 100. In the exemplary embodiment shown herein, the fastening member 108 is configured as a fastener or setscrew 130. In some embodiments, the setscrew 130 is of a non-clamping type that does not securely engage or compress against the elongate member 22 positioned within the head portion 106 of the bone anchor 100, the details of which will be discussed below. However, it should be understood that other types and configurations of fastening members are also contemplated for use for coupling the spinal rod 22 with the bone anchor 100.

In one embodiment, the setscrew 130 generally includes a body portion 132, a flange portion 134, and a head portion 136. The body portion 132 of the setscrew 130 is the portion configured to extend into the opening 120 of the bone anchor head portion 106. It includes external threads 140 configured for threading engagement with the internal threads 119 formed along the arm portions 114a, 114b of the bone anchor head portion 106. The body portion 132 also defines a lower end surface 142 facing into the U-shaped passage 110 when the setscrew 130 is engaged between the arm portions 114a, 114b, as shown in FIG. 4C. As can be seen in the example in FIGS. 4B and 5, the setscrew 130 includes only a small number of threads. In the exemplary embodiment shown, these threads may engage the screw head portion 106 with, for example, two or less than two full-thread turns. Other examples however, include more than two full thread turns. In some embodiments, the setscrew 130 includes threads 140 configured to engage the head portion 106 with threads extending less than 720 degrees about the exterior of the body portion 132. In other embodiments, the setscrew 130 includes threads 140 configured to engage the head portion 106 with threads extending less than 600 degrees about the exterior of the body portion 132, and in yet other embodiments, less than about 540 degrees. In some embodiments, to aid with reducing splay, the threads 140 may be reverse angle threads that interface with the threads on 119 on the head portion 106. Other thread styles are contemplated.

As shown best in FIGS. 5 and 6, the body portion 132 of the setscrew 130 includes an axially aligned tool engaging feature 143 for engagement with a corresponding portion of a driving tool for application of a rotary driving force to the setscrew 130. In the exemplary embodiment shown, the tool engaging feature comprises a through hole configured to receive a star-shaped tool. Other shapes however, including triangular and hex-shaped among others are contemplated. As will be discussed further below, the tool engaging feature 143 may be particularly suited for removing the setscrew 130 and the head portion 136 may be suited for inserting the setscrew 130.

In the embodiment shown, the flange portion 134 of the setscrew 130 extends outwardly from the body portion, forming a completely circular perimeter. In some embodiments however, the flange portion does not form a completely circular perimeter but the perimeter may include a straight portion, such as is formed by a line that intersects the circular perimeter in two locations. The flange portion 134 is the portion of the setscrew 130 configured to interface with the exterior portions of the bone anchor head 106. The exemplary flange portion 134 includes a transversely extending protruding portion 150 and an angled protruding portion 152. The transversely extending protruding portion 150 defines a rotational bearing surface portion 154. At the end of the transversely extending protruding portion 150, the angled protruding portion 152 defines an angled surface portion 156, best seen in FIGS. 5 and 6. Both the rotational bearing surface portion 154 and the angled surface portion 156 are sized to interface with corresponding surfaces on the head portion 106 as is shown in FIG. 4C.

In some embodiments, the angled surface portion 156 is angled relative to the longitudinal axis within a range of 30-60 degrees, and in other embodiments, within a range of 35-50 degrees. The angle of the angled surface portion 156 may be similar to the angle of the of the angled outer surface portion 124 of the head portion 106, as shown in FIG. 4C. Because of its angle, in some exemplary embodiments, the angled surface portion 156 forms a frusto-conical shape. Also, as described above, the angled outer surface portion 124 may form a frusto-conical shape.

The head portion 136 of the setscrew 130 extends from the flange portion 134 and defines a tool engaging feature 144 for engagement with a corresponding portion of a driving tool for application of a rotary driving force to the setscrew 130. In the exemplary embodiment shown, the tool engaging feature comprises an exterior surface 144 configured to interface with a driving tool. This exterior surface may be, for example, a hex or other shape suitable for driving the setscrew 130. In some embodiments, the tool engaging feature may comprise a recess configured to receive a corresponding projection of a driving tool therein. In a specific embodiment, the recess has a hexagonal configuration sized to receive a hexagonal-shaped end portion of a driving tool therein. However, it should be understood that the setscrew 130 may be provided with other types and configurations of tool engaging features that would be apparent to those of skill in the art.

In some embodiments, the head portion 136 is removably attached to the remainder of the setscrew 130 in such a manner as to allow selective separation of the head portion 136. In the illustrated exemplary embodiment, the head portion 136 is attached to the flange portion 134 by a region of reduced strength 148 to allow the head portion 136 to be fractured or broken off from the remainder of the setscrew 130 once a torque about a certain threshold is applied. In some embodiments, the region of reduced strength 148 is formed by a reduced cross-sectional area adjacent the connection location between the head portion 136 and the flange portion 134. However, it should be understood that other features for allowing selective removal of the head portion 136 from the remainder of the setscrew 130 are also contemplated as falling within the scope of the present invention. It should also be understood that the in other embodiments of the invention the setscrew 130 need not necessarily include a removable head portion 136. Instead, one or more tool engaging features may be incorporated into the body portion 132 and/or the flange portion 134 for application of a rotary driving force to the setscrew 130.

As indicated above, the setscrew 130 may be sized and configured to tighten onto the head portion 106 without applying a loading force on a longitudinal member. To accomplish this, the body portion 132 of the setscrew 130 has a height h₁ (FIG. 6) that is less than the depth d of the transverse passage 110 in the anchor head portion 106 minus the diameter of the elongate member 22 (FIG. 4B). In addition, as shown in FIG. 6, the flange portion has an overall height h₃ and the transversely extending protruding portion 150 has a thickness or height h₂. In some embodiments, the protruding portion height h2 comprises more than about half of the flange portion overall height h3, and in other embodiments, comprises about 65% or more of the flange portion overall height h3. Accordingly, the axial height h₄ of the of the angled surface portion 156 may in some embodiments be less than half of the overall height h₃ and in some embodiments, less than about 35% of the overall height h₃. In other embodiments however, the axial height h₄ of the of the angled surface is greater than the one half of the overall height h₃.

Having described the various features associated with the spinal rod 22 and the anchor members 24, reference will now be made to assembly of the stabilization system 20 and the interaction between the spinal rod 22 and the anchor members 24 accordingly to one embodiment of the invention. In the illustrated embodiment, an anchor member 24a of the fixed or clamped type is anchored to each side of the vertebra V₄, an anchor member 24b of the variable or unclamped type (e.g., bone screw 100) is anchored to each side of the vertebra V₁, and an anchor member 24c of the variable or unclamped type (e.g., bone screw 100) is anchored to each side of the vertebra V₇. However, as also indicated above, it should be understood that the anchor members may be anchored to any of the vertebrae via other layout arrangements or configurations.

Once properly anchored, an elongate member 22 (e.g., a spinal rod) is inserted into the passages 110 via the open upper ends 120 of the head portions 106. The elongate members 22 may be contoured or bent to facilitate correction of the spinal deformity and/or to accommodate for the curvature or anatomy of the spinal column.

Notably, the end portions 22a, 22b of the elongate members 22 extend beyond the upper and lower anchor members 24b, 24c a significant distance to accommodate for future growth of the patient's spinal column. In some embodiments of the invention, the end portions 22a, 22b of the elongate members 22 extend about one to two inches beyond the upper and lower anchor members 24b, 24c. However, other lengths of the end portions 22a, 22b are also contemplated.

Following insertion of the elongate members 22 into the transverse passages 110 of the anchor members 24a, a first setscrew of the clamping type is threadingly engaged to clamp the elongate member in place, thereby substantially preventing relative movement between the elongate member 22 and the clamping anchor member 24a.

Following insertion of the elongate members 22 into the transverse passages 110 of the anchor members 24b, 24c, a setscrew 130 of the nonclamping type is threadingly engaged between the arm portions 114a, 114b of each anchor member 24b, 24c. The setscrew 130 is axially advanced along the threads formed in the transverse passage 110 until the flange portion 134 is engaged against the upper end surfaces 122a, 122b and the angled outer surface portion 124 of the head portion 106 as shown in FIG. 4C.

Notably, the lower end surface 142 of the setscrew 130 is not compressed against the elongate member 22 when the rotational bearing surface portion 154 defined by the flange portion 134 is engaged against the upper end surfaces 122a, 122b of the head portion 106. Instead, a gap exists between the lower end surface 142 of the setscrew 130 and the elongate member 22 as shown in FIG. 4B. This permits relative axial movement between the elongate member 22 and the anchor members 24b, 24c. In other words, movement of the elongate member 22 is unclamped relative to the anchor members 24b, 24c. As should be appreciated, the gap is formed by providing a setscrew 130 including a body portion 132 having a height h₁ that is less than the difference between the depth d of the transverse passage 110 in the anchor head portion 106 and the diameter of the elongate member 22.

Because of its angle, the angled surface portion 156 of the flange portion 134 is configured to apply loading on the angled outer surface portion 124 of the head portion 106 in both a transverse and a longitudinal direction. This is shown in FIG. 6, where exemplary force vectors 160 show the direction of a main load F applied by the angled surface portion 156. Because of its angle, the main load F includes a longitudinal force component F₁ and a transverse force component Fₜ. The longitudinal force component F₁ aids in securing the fastening member 108 to the head portion 106 by providing a loading opposite to loading applied by the threads. The transverse force component Fₜ resists splaying of the arm portions 114a, 114b.

Once the flange portion 134 of the setscrew 130 is tightly compressed against the upper end surfaces 122a, 122b of the head portion 106, exerting additional torque onto the setscrew 130 beyond a predetermined level will cause the head portion 136 to be sheared or broken off from the remainder of the setscrew 130 adjacent the region of reduced strength 148. After the head portion 136 of the setscrew 130 is removed, the only portion of the setscrew 130 extending beyond the upper end surfaces 122a, 122b of the arm portions 114a, 114b is the flange portion 134.

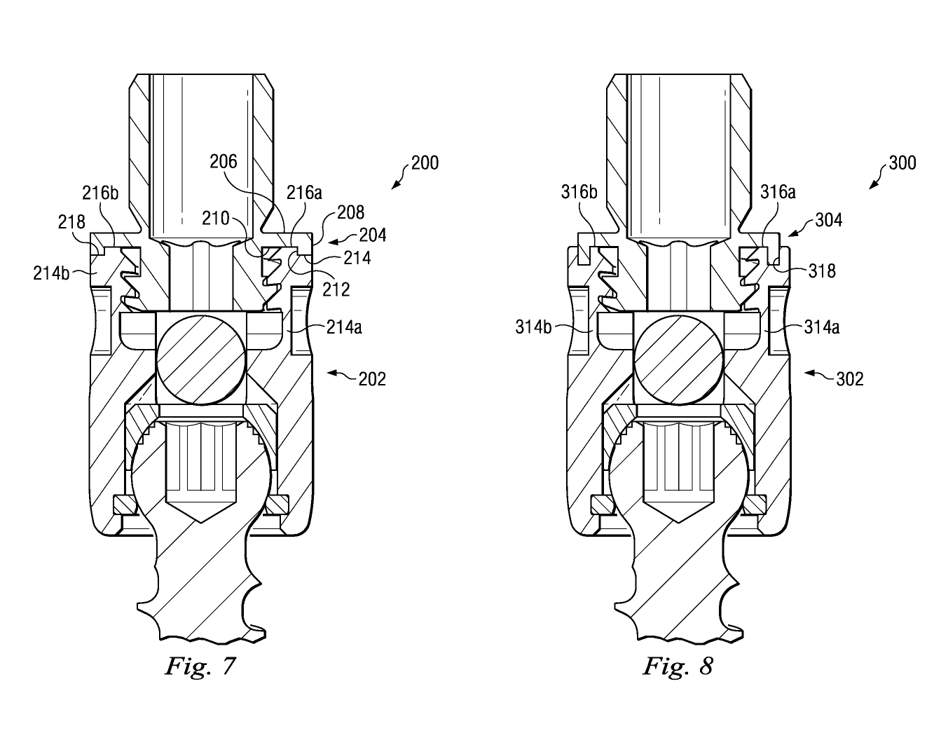

FIGS. 7 and 8 show alternative embodiments of anchor members with fastening members that include a flange portion having an angled portion angled at about 90 degrees and with alternative head portions.

Turning to FIG. 7, an alternative fastening member 200 and an alternative head portion 202 are shown together in cross-section. Both the fastening member 200 and the head portion 202 include many of the same features discussed herein with reference to other embodiments. For simplicity, only the differences between the embodiment in FIG. 7 and the earlier embodiments will be discussed.

In the exemplary embodiment of FIG. 7, the fastening member 200 includes an alternative flange portion 204. As can be seen, the flange portion 204 includes a transversely extending protruding portion 206 and an angled protruding portion 208. The transversely extending protruding portion 206 defines a rotational bearing surface portion 210. At the end of the transversely extending protruding portion 204, the angled protruding portion 208 defines a transverse angled surface portion 212 and a longitudinal angled surface portion 214. The rotational bearing surface portion 210, the transverse angled surface portion 212, and the longitudinal angled surface portion 214 are sized to interface with corresponding surfaces on the head portion 202.

As shown in the cross-section the head portion 202 includes a pair of spaced apart arm portions 214a, 214b having ends 216a, 216b. Adjacent the ends 216a, 216b, the head portion 202 includes an indent portion 218. The indent portion 218 is formed in the exterior surface of the head portion 202 and sized to receive the angled protruding portion 208 of the fastening member 200. Accordingly, in this embodiment, the rotational bearing surface portion 210 is configured to apply loading against the ends 216 of the fastening member 200. In addition the transverse angled surface portion 212 is configured to apply loading in a direction that reduces the chance of splaying, and the longitudinal angled surface portion 214 is configured to apply loading in the same direction as the rotational bearing surface portion 210. In some embodiments, it is contemplated that the longitudinal angled surface portion 214 is arranged in a manner to not apply loading to the indent portion 218. Other arrangements also are contemplated.

Turning to FIG. 8, an alternative fastening member 300 and an alternative head portion 302 are shown together in cross-section. Both the fastening member 300 and the head portion 302 include many of the same features discussed herein with reference to other embodiments. For simplicity, only the differences between the embodiment in FIG. 8 and the earlier embodiments will be discussed.

In the exemplary embodiment of FIG. 8, the fastening member 300 includes an alternative flange portion 304 that is similar in many ways to the flange portion 204 discussed with reference to FIG. 7. The duplicative features are not discussed here.

The head portion 302 includes a pair of spaced apart arm portions 314a, 314b having ends 316a, 316b. Adjacent the ends 316a, 316b, the head portion 302 includes a groove portion 318. The groove portion 318 is sized to receive the angled protruding portion of the fastening member 300. Here, however, the groove is not formed at the exterior surface of the head portion 302. Instead, it is formed in the end surfaces 316a, 316b. Accordingly, a portion of the flange member 304 is received within the groove 318 and not on the exterior of the head portion 302.

As should become apparent, by employing flanges on the fastening members usable with the head portion members of nonclamping closure mechanisms, splay of the anchor members may be reduced without increasing the overall height of the head portion. This reduces the chance of revision surgery to correct a splaying head without increasing the patient discomfort that may arise from overly tall head portions.

While embodiments of the invention have been illustrated and described in detail in the disclosure, the disclosure is to be considered as illustrative and not restrictive in character. All changes and modifications that come within the spirit of the invention are to be considered within the scope of the disclosure.

We claim:

1. A device for attaching an elongate support member to the spinal column, comprising:
a head portion having a U-shaped portion defining a transverse passage therethrough, the passage being sized to receive a portion of the elongate support member, the head portion having an opening between a pair of spaced apart arm portions, the arm portions having end surfaces and having inwardly facing threads; and
a fastening member comprising:
a body portion having threads sized to interface with inwardly facing threads on the head portion, the body portion defining a longitudinal axis; and
a flange portion adjacent the body portion, the flange portion extending outwardly about the longitudinal axis to define a circular flange perimeter, the flange portion including a transversely extending protruding portion and an angled protruding portion.

2. The device of claim 1, wherein the head portion comprises a groove in the end surfaces, the angled protruding portion being sized to fit into the groove in the end surfaces.

3. The device of claim 1, wherein the angled protruding portion includes an angled surface portion configured to interface with the head portion in a manner that applies loading to the head portion in both a longitudinal direction and a transverse direction.

4. The device of claim 1, wherein the angled protruding portion forms a 90 degree angle relative to the transversely extending protruding portion.

5. The device of claim 1, wherein the angled surface portion of the fastening member defines a frusto-conical shape.

6. The device of claim 1, wherein the flange is configured to apply loading to at least one of the end surfaces of the head portion.

7. A setscrew for a spinal construct, the spinal construct including a head portion, comprising:
a body portion having threads sized to interface with inner threads on the head portion, the threads extending less than 720 degrees about the exterior of the body portion, the body portion defining a longitudinal axis; and
a flange portion adjacent the body portion, the flange portion extending outwardly about the longitudinal axis to define a circular flange perimeter, the flange including a transversely extending protruding portion and an angled protruding portion.

8. The setscrew of claim 7, further comprising a break-off type head portion comprising a surface configured to cooperate with a driving tool.

9. The setscrew of claim 8, wherein the angled protruding portion forms a 90 degree angle relative to the transversely extending protruding portion.

10. The setscrew of claim 7, wherein the angled protruding portion includes an angled surface portion configured to interface with the head portion in a manner that applies loading to the head portion in both a longitudinal direction and a transverse direction.

11. The setscrew of claim 7, wherein the threads extend less than about 600 degrees about the exterior of the body portion.

12. An anchor member for attaching an elongate support member to the spinal column, comprising:
a head portion defining a first longitudinal axis and having a transverse passage therethrough configured to receive a portion of said support member; and
a setscrew member threadably engageable with the head portion in a manner to secure the support member the passage, the setscrew member defining a second longitudinal axis and having a transversely extending flange, the flange having a surface portion configured to interface with the head portion in a manner that applies loading to the head portion in both a longitudinal direction and a transverse direction.

13. The device of claim 12, wherein the surface portion of the setscrew member defines a frusto-conical shape.

14. The device of claim 12, wherein the head portion includes an end surface, the flange being configured to apply loading to the end surface.

15. The device of claim 12, wherein the loading in the transverse direction inhibits splay and the loading in the longitudinal direction helps secure the setscrew to the head portion.

16. The device of claim 12, wherein the head portion includes an outer surface portion angling at a first angle and the surface portion of the flange is angled to have a similar angle.

17. The device of claim 12, wherein the setscrew member includes less than three full turns of thread.

18. The device of claim 12, wherein the setscrew is sized so that the support member in the passage is non-rigidly secured in the passage of the head portion.

19. The device of claim 12, wherein the flange has a complete circular circumference.

20. The device of claim 12, wherein the flange includes a transversely protruding surface portion and an angled surface portion.

21. The device of claim 12, wherein the setscrew is a break-off type setscrew.

22. An implant, comprising:
an elongate support member sized to span a plurality of vertebrae;
an anchor member configured to provide substantially unconstrained axial movement of the elongate member relative to the anchor member, the anchor member including:
a bone engaging portion; and
a head portion connected to the bone-engaging portion, the head portion having a U-shaped portion defining a transverse passage therethrough sized to receive a portion of the support member, the head portion having a frusto-conical outer surface portion; and
a setscrew member threadably engageable with the head portion, the setscrew member defining a longitudinal axis and having a transversely extending flange, the flange having a frusto-conical surface portion configured to interface with the frusto-conical outer surface portion of the head portion, the setscrew being sized to engage with the head portion in a manner that non-rigidly secures the support member in the passage when the flange portion interfaces with the head portion.

23. A device for attaching an elongate support member to the spinal column, comprising:
a head portion having a U-shaped portion defining a transverse passage therethrough, the passage being sized to receive a portion of the elongate support member, the head portion having an opening between a pair of spaced apart arm portions, the arm portions having end surfaces; and
a fastening member comprising:
a body portion having threads sized to interface with the head portion, the body portion defining a longitudinal axis; and
a flange portion adjacent the body portion, the flange portion extending outwardly about the longitudinal axis to define a circular flange perimeter, the flange portion including a transversely extending protruding portion and an angled protruding portion.
